(19) United States
(12) Patent Application Publication
(10) Pub. No.: US 2007/0141170 A1
(43) Pub. Date: Jun. 21, 2007

(54) COMPOSITION AND METHODS FOR INHIBITING THE PROGRESSION MACULAR DEGENERATION AND PROMOTING HEALTHY VISION

(75) Inventor: JOHN C. LANG, Cedar Hill, TX (US)

Correspondence Address: ALCON, IP LEGAL, TB4-8, 6201 SOUTH FREEWAY, FORT WORTH, TX, 76134, US

(73) Assignee: ALCON MANUFACTURING, LTD., FORT WORTH, TX (US)

(21) Appl. No.: 11/613,938

(22) Filed: Dec. 20, 2006

Related U.S. Application Data
(60) Provisional application No. 60/751,836, filed on Dec. 20, 2005.

(51) Int. Cl. A61K 33/34; A61K 31/355; A61K 31/195; A61K 31/525; A61K 31/51; A61K 33/26; A61K 33/04; A61K 36/53

(52) U.S. Cl. 424/638; 424/641; 424/745; 424/646; 424/702; 424/722; 514/52; 514/251; 514/168; 514/276; 514/350; 514/356; 514/393; 514/561; 514/474; 514/458; 514/763

(57) ABSTRACT

The present invention provides improved dietary supplements and methods for inhibiting the progression of macular degeneration and promoting healthy vision, while at the same time maintaining general health. The dietary supplements of the invention contain vitamin E and carotenoids in the form of Vitamin A, lutein and/or zeaxanthine. The dietary supplements of the invention further contain Vitamin C, copper and zinc and may also contain such ingredients as rosemary, DHA, and other vitamins and minerals.

This application claims priority to U.S. provisional application Ser. No. 60/751,836 filed Dec. 20, 2005.

### BACKGROUND OF THE INVENTION

1. Field of the Invention

The present invention generally relates to nutritional methods and compositions for alleviating eye diseases and, more specifically, to improved methods and compositions for the treatment of cataracts and macular degeneration.

2. Description of the Related Art

Macular degeneration, associated with aging and appearance of drusen, is an extremely significant concern, for AMD (age-related macular degeneration) is now a major cause of blindness in the United States for individuals over 65 years of age. Just at the period of time when the eyes are a most important sense, and reading and watching television are often the most enjoyable avenues of entertainment, this disease robs the elderly patient of such possibilities.

The crystalline lens of the eye has only one disease state that we are aware of, and that is cataract. The lens loses its clarity as it becomes opacified, and vision is disturbed depending on the degree of opacification. There are different etiologies for cataracts such as a congenital lesion or trauma, which are well recognized. It is also known that some medicines such as cortisone-type preparations and glaucoma medications can cause cataracts, as can early onset metabolic errors such as galactosemia or latent genetic errors resulting in diabetes. These, however, are less common than the more familiar age-related cataract, which is associated with the cumulative oxidative stress that results in cross-linked and precipitated protein.

The exact incidence of cataracts in the general population is difficult to determine because it depends in part on one's definition of a cataract. If defined as simply a lens opacity, then obviously the incidence is much higher than when defined as a lens opacity that significantly impacts vision. The pathogeneses of age-related cataracts and macular degeneration are incompletely understood.

The accumulation of drusen and lipofuscin and the loss of retinal pigment, hallmarks of macular degeneration, appear to be a consequence of the accumulation of biomolecular derivatives of bioactive molecules involved in photoreception and signal processing, and normally detoxified, processed, and exported from the RPE (retinal pigment epithelium). While the importance of controlling the accumulation of lipofuscin and its dominant toxic component A₂E, N-retinylidene-N-retinylethanolamine (Sparrow, 2001), which is capable of converting visible-wavelength radiation into toxic ROSs (reactive oxygen species), is acknowledged, no means for accomplishing this has been proposed and so one of the best means currently available for limiting the damage is by reducing the amount of radiation available to the lipofuscin. There also is no effective treatment to date for the resulting atrophy or angiogenesis, except attempted laser photocoagulation in those patients who develop abnormal blood vessels under the retina, i.e., subretinal neovascularization. The treatable group with advanced AMD is a distinct minority of a much larger group. Individuals so afflicted can anticipate either a progressive deterioration or at times relatively static course, but no spontaneous improvement, since the basic architecture of the retina is destroyed. Occasionally, there may be variations in vision which seem to show improvement depending on such things as lighting in the room and potential resolution of fluid underneath the retina. The important point, however, is that when this sensitive neurological tissue is damaged, that damage is permanent.

In 1981, Spector et al. stated that there still remained questions concerning the mechanism and agents involved with massive oxidation of the lens proteins and its relationship to cataract development (Spector et al. 1981). They also noted that glutathione (GSH) can act as a reducing agent and free radical trapper. Glutathione peroxidase (GSHPx) and catalase are present to metabolize H₂O₂. While superoxide dismutase (SOD) can detoxify O₂, light can photochemically induce oxidation. However, Spector et al. believe that while the complete mechanisms of light and/or metabolically-induced oxidation are unclear as to causing the observed oxidation products, they appear to be associated with elevated levels of intracellular oxidizing agents, such as hydrogen peroxide.

In 1987, Machlin et al. reported that there was some evidence that free radical damage contributed to the etiology of some diseases, including cataract (Machlin et al. 1987). They indicated that defenses against such free radical damage included Vitamin E, Vitamin C, beta carotene, zinc, iron, copper, manganese, and selenium.

In 1988, Jacques et al. reported that it is commonly believed that oxidative mechanisms are causally linked to, not simply associated with, cataract formation. According to Jacques et al. evidence suggests that GSHPx and SOD decrease with increasing degree of cataract.

Jacques et al. further reported that Vitamin E is believed to be a determinant of cataract formation and can act synergistically with GSHPx to prevent oxidative damage. They point out the possibility that Vitamin C may have a role in cataract formation and might influence GSHPx through its ability to regenerate Vitamin E.

Dietary supplements are taken for a variety of reasons including the improvement of vision or prophylaxis against vision loss. An example of a set of dietary supplements useful in promoting healthy eyes are the ICAPS® Dietary Supplements (Alcon Laboratories, Inc., Fort Worth, Tex.). Dietary supplements are generally in the form of powders, tablets, chewable tablets, capsules, gel-caps or liquid-fill softgels and comprise a variety of vitamins, minerals, and herbal or other organic constituents. Some dietary supplements are formulated with beadlets.

Recent data have suggested that the inclusion of xanthophylls and other carotenoids in dietary supplements may provide superior dietary supplements useful in enhancing the health of the eye. Studies have shown selective uptake of the carotenoids, zeaxanthin and lutein, by the retina at the ratio of about 2:1 for lutein:zeaxanthin but with the ratio inverting in the macula (Bernstein et al. 1997 & 2004; Bone and Landrum et al. 1988 & 2001; Krinsky et al 2003; Hammond et al. 1997; and Handelman et al. 1991). This earlier work revealed the presence of both lutein and its positional isomer, [R,R]-zeaxanthin. More recently, a second isomer of zeaxanthin has been found in the macula, the diastereomer meso-zeaxanthin, the [R,S] isomer of zeaxanthin (Bone & Landrum et al. 1988). These and related observations suggest both are essential for improved ocular health and protection of the macula.

Xanthophylls are effective phytochemical antioxidants and are known to localize in the macula of the retina. It has been suggested that the particular xanthophylls, zeaxanthin and its isomer lutein, may be beneficial in maintaining or improving the health of the macula and the clarity of the lens. These molecules may function in a number of ways to protect the eye from high intensity radiation or other insults. It has been suggested that foveal proteins bind the xanthophylls, localize and concentrate xanthophylls within the fovea (Bernstein et al. 2004). Since xanthophylls are capable of absorbing photoexcitative radiation of short visible wavelength, they also may shield the light-sensitive, underlying cells of the neural retina and RPE. Such cells are responsible for high-definition vision and have been shown by epidemiological studies to be adversely affected by exposure to high intensity radiation or even chronic exposure to visible wavelength radiation. The carotenoids are believed to complement the activity of these cells, and also to protect them against photochemical insult. See, e.g., Snodderly (1995) and Seddon et al. (1994).

Studies also have shown that the portion of the retina associated with xanthophyll deposition undergoes one of the highest metabolic rates in the body (Berman 1991). The energy to sustain this metabolism is derived from oxidation. While the very lipophilic xanthophylls do not appear to undergo rapid turnover characteristic of water-soluble or surface active antioxidants (Hammond et al. 1997), continuous exchange of xanthophylls occurs in response to both environmental challenge and tissue environment, and their gradual depletion without nutritional replacement may portend tissue damage (Hammond et al. 1996a; Hammond et al. 1996b; and Seddon et al. 1994). The lack of rapid turnover also implicates the role of other synergistic antioxidants, vitamins C and E, especially but also enzymatic antioxidants that are active in the redox cascade that passes the initial oxidative excitation to lower-energy and less damaging species.

The carotenes are conjugated C₄₀ compounds that include beta carotene (a provitamin, a vitamin A precursor). The carotenes are deeply colored compounds and are found throughout the plant kingdom, e.g., in leafy vegetables such as spinach and kale, and brilliantly colored fruits such as melons and pineapple. While the carotenes are ubiquitous in the plant kingdom, they generally are not available biosynthetically in mammals. Since the carotenes are essential for normal mammalian health, mammals need to ingest various sources of the carotenes, e.g., fruits and vegetables. The absence of carotenoids from the diet, especially the carotene derivative, vitamin A, is known to be associated with degenerative eye diseases.

Another important component for maintaining the health of the elderly or aging patient is insuring intake of appropriate amounts of vitamins and minerals. Because of compromised bioabsorptive capacity, many elderly and aging patients are unable to ingest the recommended amount of vitamins and minerals through diet alone. Moreover, aging patients tend to be on a number of prescription medications as well. Remembering to take all prescribed medications at the appropriate time every day can prove to be a challenge to the elderly patient. Adding a multi-vitamin and another dietary supplement for ocular health increases the chances of non-compliance with intake of daily medications. Needed for an elderly and aging population is a single dietary supplement that provides both the recommended daily amount of vitamins and minerals while at the same time providing supplementation with additional vitamins, minerals, and essential nutrients at levels recommended for maintaining ocular health.

### SUMMARY OF THE INVENTION

The present invention overcomes these and other drawbacks of the prior art by providing a multi-vitamin dietary supplement containing recommended dietary amounts, or above, of a number of necessary/essential vitamins and minerals for general body health along with a unique combination of additional vitamins, minerals, and essential nutrients necessary for maintaining or improving ocular health.

The present invention is directed to improved formulations useful for maintaining and improving both ocular and systemic health. In particular, the improved formulations comprise specific combinations and amounts of vitamins and minerals proven in the Age-Related Eye Disease Study (AREDS) to slow progression of AMD, with multivitamin, mineral and essential nutrient components to maintain the general health of the patient. Such improved formulations may additionally provide lutein and zeaxanthin in the ratio shown to be present in the retina. Preferred formulations may also contain one or more bioflavonoids and other phytonutrients providing antioxidant or signaling and control functions to protect ocular tissues from detrimental metabolites generated by photo-oxidative stress.

The advantage of the specific combinations of ingredients is that they are essentially complete, and are selected to eliminate imbalances of ingredients that may occur when multiple products are combined. In addition, different versions are claimed that are specialized for different segments of the population, segments which may have specific dietary requirements or restrictions.

### DETAILED DESCRIPTION PREFERRED EMBODIMENTS

According to the present invention, the elements of the composition are directed toward scavenging free radicals and oxidants or in other ways retarding disease progression of macular degeneration. At the same time, the formulations of the present invention provide components of a multi-vitamin needed by the elderly patient in order to maintain general health. The free radicals to which the present invention is directed primarily include superoxide and the hydroxide free radical. The oxidants include primarily peroxide.

The items and doses in the present invention are consistent with those readily available in health food stores. The dosage form is preferably a tablet, caplet or softgel form for oral administration, with the patient taking one to four doses taken once or twice a day. The present invention, however, contemplates that the preferred total dosage can be administered as a single dose or other multiple part dosages. The composition may also be of the timed-release or delayed-release types. Further, for oral administration, the present composition may be in capsules, lacquered tablets, unlacquered tablets, softgels, or blends of controlled release powders, prepared according to well-known methods. In accordance with the preferred multiple dosages described above, each tablet, caplet, or softgel is preferably composed approximately as follows:

### Vitamin C

It has been known that there are high concentrations of Vitamin C both in the normal human lens and in the aqueous humor that surrounds the lens, and that this is an antioxidant (Harris 1933). It has also been shown in the past that generally increasing dietary Vitamin C generally increases the concentration of ascorbate in the aqueous humor and in the human lens (Ringvold 1985). It has also been known that Vitamin C concentrations decrease with age and, in particular, in patients who have senile cataract (Chatterjee 1956; Purcell 1968). Subsequent work has demonstrated that supplementation with Vitamin C is effective in increasing lens concentrations of this water-soluble antioxidant, and epidemiological data support its value for reducing the prevalence of cataract (Taylor, 1999). It also has been shown that Vitamin C is integral to the antioxidant cascade that reduces oxygen to water, capable of regenerating the reduced form of Vitamin E, localized in biomembranes.

There is no known optimal daily dose of Vitamin C, although the U.S. RDA is 60 mg. However, dosages of 2.0 grams and more have frequently been taken as a supplement for general health. Although ascorbic acid or rose hips can be used, the present composition preferably utilizes Vitamin C in the form of sodium ascorbate because of its being easily dissolved in the digestive system and causing relatively minimal irritation. The concentration is at about 200-250 mg/tablet or caplet, or a preferred total dosage of about 0.8-2 grams/day. In such concentrations, the Vitamin C represents about 20-30% by weight of each tablet or caplet, which includes active as well as inactive ingredients described below.

### Vitamin E

Vitamin E is also a well-known antioxidant, as already mentioned (see also Mansour 1984). Vitamin E can work synergistically with Vitamin C in protecting vital cell function from endogenous oxidants (Orten 1982).

A very common Vitamin E supplementation consists of 400 International Units per day. While studies that used more than 800 IU per day have shown possible signs of toxicity, many common dietary supplements available in supermarkets have 1000 units of Vitamin E daily (e.g., Chaney 1986). The U.S. RDA is 30 IU. The present invention preferably uses Vitamin E in the form of d,1-alpha tocopheryl acetate, for which 1 mg is equivalent to 1 IU. The preferred concentration is about 15 IU-400 IU per tablet or caplet or a total daily dosage of 30-800 IU of Vitamin E. This represents from about 1% to preferably less than 20% by weight of each tablet or caplet.

### Zinc

Zinc is known to be important to the health of the retina and the function of Vitamin A (Russell 1983; Karcioglu 1982; Leure-duPree 1982). Zinc is a cofactor in an enzyme required for maintaining the bioavailability of folate (Chandler et al. 1986), and folate is important for healthy DNA and protein synthesis. Zinc is one supplement previously used in a study which showed it to be significantly better than placebo in retarding macular degenerative changes (Newsome 1988). Zinc is also known to be an important cofactor for a whole multitude of metalloenzymes, not the least of which is superoxide dismutase, which scavenges the potent oxidizer—superoxide. There are two types of SOD in mammalian cells. One type contains copper and zinc and is located in the cytosol and periplasmic space of the mitochondria. The other type contains manganese and is in the matrix of the mitochondria (see generally U.S. Pat. No. 4,657,928). Mitochondria are the site of the high metabolic activity, and rapid oxidative processes in cells of the neural retina and retinal pigment epithelium (RPE), providing the energy needed for converting the stimulus of visible light radiation to a chemical signal. These isoforms of SOD and zinc are also implicated in cataract because both superoxide dismutase activity and zinc are dramatically lower in cataract patients than in noncataract patients (Ohrloff 1984; Varma 1977; Swanson 1971). Zinc is also involved in enzymes related to the metabolism of vitamin A, regulating the levels of esterification. By so doing, zinc is implicated in regulating hepatic storage, release, and transport of retinol, and thereby its bioavailability for ocular tissues (Russell 1983).

About 200 mg of zinc per day, although well-tolerated, has been shown to have potential side effects, particularly blocking copper absorption, which results in the possibility of copper deficiency anemia (Fischer 1983). High doses also have been shown to have the effect of lowering serum titer of high-density lipoprotein, thereby potentially exacerbating the risk of atherosclerosis (Hooper 1980).

The dosages of 100-150 mg of zinc a day have been known in the past to be well tolerated without difficulty (Wagner 1985). The U.S. RDA is 15 mg. While other salt forms such as sulfate, picolinate, phosphate, and gluconate can be used, the present invention preferably provides the zinc in the form of zinc acetate because of its high bioavailability, and zinc oxide because of its high density of zinc. The preferred daily dosage range is from the RDA to a maximum of about 100 mg of a bioavailable form of zinc, such as zinc acetate. This maximum amount of zinc in a less bioavailable form such as zinc oxide could range as high as 150 mg/day. Either form could be administered in a tablet, caplet, powder or softgel.

### Copper

Copper is another important cofactor for metalloenzymes, and is a second necessary cofactor for superoxide dismutase (Beem 1974). Copper has been shown to decrease in individuals over 70 years of age and to be basically zero in cataractous lenses (Swanson 1971). If copper is significantly decreased, superoxide dismutase has been shown to have decreased function, thereby hampering an important mechanism for protecting the lens (Williams 1977). Copper is also protective of zinc toxicity, which blocks some of the zinc absorption and, therefore, decreases bioavailability (Van Campen 1970).

Two to three mg of copper per day have been estimated to be safe and provide adequate daily dietary intake (Pennington 1986). A two mg daily dose is the U.S. RDA. Some copper absorption will be blocked by the 100 mg of daily zinc as provided above (Van Campen 1970). Therefore, the present composition preferably provides about 1-5 mg/day. This amount is considered safe because in the typical American diet, particularly among the elderly, zinc and copper are often significantly below minimum daily requirements. In this embodiment of the present invention, copper is provided preferably in the form of copper gluconate, citrate, or an amino acid chelate and copper in such form typically represents less than about 3% by weight of each tablet or caplet for a typical BID administered supplement like ICaps® Lutein and Zeaxanthin Formula, and less than 1% for a typical QID administered supplement like ICaps® AREDS. Cupric oxide also has been utilized as a source of copper in supplements where the total available space in the dosage form is very limited, since the fraction of copper is higher in this compound.

### Beta-Carotene

It is well-known that Vitamin A is essential for vision. Vitamin A, retinol, is a C₂₀ alkene, which as retinal is combined with opsin in the retina to form rhodopsin, a visual pigment. The transition of the cis form to the trans form of retinal results from excitation by light. Thus, clearly vitamin A is crucial to photoreception. Beta-carotene, a pro-vitamin A carotenoid, is a lipid-soluble orange pigment that can serve as a self-regulating source of retinal. Both deficiency and excess of retinol can lead to fetal abnormalities since vitamin A is associated with not only vision but also growth, reproduction, cell proliferation, cell differentiation, and proper immune function.

The amount of β-carotene converted to retinol is biologically controlled and dictated by the need for retinol. The control is exerted through the central symmetric enzymatic cleavage of the C₄₀-carotenoid to the C₂₀-retinoid. Therefore, none of the types of vitamin A toxicity have been observed for β-carotene. Nonetheless and surprisingly, explicit β-carotene toxicity has been unearthed. While treatment of a β-carotene deficiency reduced the incidence of esophageal and gastric cancers, a compromised handling of a xenobiotic was seen in connection with its use in treating lung cancer and cardiovascular disease in smokers given high daily doses (i.e., 30 mg/day) of β-carotene. As a consequence, smokers (a high risk category for AMD) are encouraged not to increase their supplemented level of β-carotene above the RDA level. This recommendation directly contradicts the recommendation coming from the 7-year ARED Study, in which about 17-24 mg/day were consumed (AREDS Research Group 2002).

The resolution of these conflicting recommendations, as prescribed below, is to provide versions of a complete formulation, including the vitamins and minerals of a multivitamin consumed by two-thirds of those on the ARED study, maintaining the total carotenoids at the 15 mg, or lower, designated level. In one formulation, lutein and zeaxanthin are substituted for a portion of the β-carotene content, maintaining the daily dosage of β-carotene at the RDA, 3 mg per day. In another, lutein and zeaxanthin replace the β-carotene entirely. The amount per tablet will be based on the number of tablets recommended for the particular dosage form, generally two to four tablets per day.

### Xanthophylls

While Xanthophylls also are C₄₀ compounds, and are carotenoids, this subclass is distinguished by the presence of more polar groups. The lutein and zeaxanthin isomers have hydroxyl alcoholic groups on both ionone terminal rings, and this plays a profound role on the localization and use of these carotenoids. Binding proteins specific to these lipids appear to control their localization in the eye, both their total absolute amount and their relative amounts. For example, observations in both primates and humans (cadaver eyes, for example) have indicated that while lutein is the most abundant xanthophyll in the eyes, in the vicinity of the fovea the relative amount of zeaxanthin is greater than lutein. The xanthophylls all serve as antioxidants, quenchers of free radicals, and absorbers of blue light, and all of these are protective functions of these molecules for the underlying retina and its support tissue, the RPE. These xanthophylls are all isomers of one another; the zeaxanthins have one more of the double bonds in the conjugated sequence, and so lutein and zeaxanthin are positional isomers. And the two zeaxanthin isomers, 3,3′-[R,R] and 3,3′-[R,S] (the meso form) are diastereomers, differing at only one optical center. All three of these diols have been observed to be present in the macula.

Xanthophylls are typically considered to be very safe compounds, found in edible plants and vegetables, from melons to corn to spinach and kale. Epidemiology has shown the incidence of AMD is lower for those individuals consuming amounts in the higher quartiles and quintiles. GRAS status has been granted to lutein, in both the free alcohol and ester forms, and to zeaxanthin, in the free alcohol form. Lutein appears interconvertible to the meso form of zeaxanthin, though the protein(s) responsible for the interconversion have not yet been identified and so the precise mechanisms and means of controlling the interconversion are unknown. As a consequence, some balance of these xanthophylls in both diet and supplementation appears most prudent.

Both epidemiologic and prospective clinical studies indicate that higher macular levels of xanthophylls protect the retina from oxidative stress. Some data support an increased deficit in the middle-aged and elderly. From epidemiologic data it was discerned that levels above 6 mg/day of xanthophylls were beneficial in delaying onset of AMD. Studies of the impact of diet on bioavailability suggest serum levels of xanthophylls increase within a period of about four to eight weeks, and macular pigment levels respond more slowly but generally within four to six months, probably dependent on age, sex, and other health and risk factors of the subject. These data also suggest that both the rate of increase and the plateau levels are dependent on the daily intake, as well as other individual factors. The National Health and Nutrition Examination Survey (NHANES) levels, that is the normal domestic U.S. intake, is about 2 mg/day. Thus, in the methods and compositions of the present invention, the total daily supplementation of xanthophylls is preferably in the range from 2 mg/day to 18 mg/day, more preferably less than about 16 mg/day.

The ratio of lutein to zeaxanthin in the retina has been shown to be about 2:1. It is believed that providing a similar ratio of lutein to purified zeaxanthin in a dietary supplement is more effective in maintaining ocular health than providing a much higher amount of lutein, such as that which may occur naturally in plant sources for the compound. Therefore, in preferred aspects of the present invention, lutein and zeaxanthin will be present in the formulation in a ratio of 2:1. For example, if there are 4 mg of lutein in the formulation, there will be 2 mg of zeaxanthin in the formulation. Likewise, 8 mg of lutein corresponds to 4 mg of zeaxanthin, and so on.

The present invention is directed to improved dietary supplement formulations for maintaining the general and ocular health of a patient or consumer. As used herein, “dietary supplement(s)” or the shortened form, “supplement(s),” refer to any finished, dietary supplement dosage form containing dietary substances and suitable for ingestion by a host, e.g., human or other mammal. Thus, the term “dietary supplement” is meant to encompass any form of dietary supplement, such as the tablet, chewable tablet, caplet, gelcap, powder, softgel, etc.

As used herein, “xanthophylls” refer to hydroxy- and keto-oxidized carotenes and their derivatives, including both free alcohols and esters; “carotenes” refer to any of the 40-carbon carotenes and their derivatives; “retinoids” refers to the 20-carbon Vitamin A (retinol) and its derivatives; and “carotenoids” refers to any of the xanthophylls, carotenes and retinoids or combinations thereof. Carotenoids may be synthetically derived or purified from natural sources. Synthetic preparations may contain different isomers of carotenoids than those contained in the natural preparations. Depending on intended use, natural, synthetic or mixtures of both types of carotenoids may be included as oils, cakes, encapsulated oils or blends, or monolithic cobeadlets in the present invention.

The xanthophyll component may be obtained from various sources such as vegetables and herbal components, such as corn, leafy green vegetables and marigolds; marine sources, such as krill; or microorganic sources, such as algae and gene-engineered bacterial or yeast sources. Xanthophylls may also be synthesized by methods known in the art and are available from various manufacturers. Examples of xanthophylls include, but are not limited to, lutein, zeaxanthin, astaxanthin, canthaxanthin, cryptoxanthin and related oleoresins (e.g., fatty acid mono and di-esters of xanthophylls). The xanthophyll purity and concentration in the various commercial sources will vary. For example, some sources may provide about a 1% weight/weight (“w/w”) or less of xanthophyll in oil while other sources, e.g., Kemin Laboratories, Inc. (Des Moines, Iowa), may provide a source in excess of 20% w/w xanthophyll in oil, or upwards of 50% as provided in the crystalline or semicrystalline ‘cake’. Xanthophyll sources may be preparations of individual xanthophylls or combinations thereof, and may range in concentration depending on the diluent, or in fact their absence since some preparations of powder or ‘cake’ may provide a more preferable raw material. For example, a xanthophyll preparation may comprise lutein as the sole xanthophyll or a combination of lutein and zeaxanthin, including combinations of the diastereomers of zeaxanthin ([R,R′], [R,S], [S,R], and [S,S]), wherein preferred combinations include a mixture of lutein, [R,R′]-zeaxanthin and meso-zeaxanthin. Other preferred combinations include a mixture of [R,R′]-zeaxanthin and meso-zeaxanthin and/or a mixture of lutein and any one diastereomer of zeaxanthin. The inclusion of a combination of xanthophylls in the formulations, and in particular ratios, may be particularly important when it is the intention to deliver such combinations to the host in ratios similar to those found in the retina broadly, or in the macula or fovea of the eye, specifically, or in other ratios which, when ingested, support the ratios in the host tissues. Xanthophylls may also be included in the formulations as conjugated derivatives, e.g., oleoresins of xanthophylls, as exemplified above.

### Omega-3 Fatty Acids

Omega-3 fatty acids, found naturally and in abundance in tissue of cold water fish, are also abundant in the optic discs of photoreceptors in human retina. Epidemiologically, it has been found that the prevalence of AMD is higher for individuals with diets depleted in omega-3 fatty acids, that is, that the amount of omega-3 in the diet correlates inversely with the prevalence of AMD (Seddon and Willett et al.). The two predominant omega-3 fatty acids, conjugated fatty acids, important in eye health are DHA (docosahexaenoic acid) and EPA (eicosapentaenoic acid). The term “DHA” as used herein refers to either of these two predominant omega-3 fatty acids or to a mixture of the two; that is, when the term “DHA” is used, the skilled artisan would understand that either DHA, EPA, or a mixture of EPA and DHA could be used in that instance. The preferred ratio of EPA to DHA when a mixture is used is 0.8:0.2 to 0.2:0.8, EPA:DHA. While docosahexaenoic has been made available from fermentation and biotechnology sources, the preferred blend is usually harvested from fish and then purified/deodorized.

### Other Vitamins/Minerals for Systemic Health

Vitamins:

Vitamin D is a primary regulator of calcium homeostasis and is essential for normal bone, muscle, and nerve growth and function. Vitamin D has been shown to protect against osteoporosis, and to have anticarcinogenic and antioxidant activities in the body. The RDI for Vitamin D has been established to be 400 IU/day.

Vitamin K is involved as a cofactor in the regulation of hemostatic proteins essential for proper blood clotting, preventing excessive bleeding. The RDI for Vitamin K has been established to be 80 μg/day.

Thiamin (Vitamin B₁) is essential for utilization of carbohydrates and fats to produce energy and support cellular metabolism. Thiamin is important in neuromuscular development and maintenance. Vitamin B₁ has been shown to have antioxidant effects in neural tissues including the brain. The RDI for thiamin has been established to be 1.5 mg/day.

Riboflavin (Vitamin B₂) is important in maintaining energy production and metabolic processes involving carbohydrates, fats and proteins and for normal cell function and growth. Riboflavin may help preserve healthy eyes, nerve and skin function. The RDI for riboflavin has been established to be 1.7 mg/day.

Niacin (Vitamin B₃) is involved in a wide array of biochemical reactions including energy production and the synthesis of fats and steroids. Vitamin B₃ has been found to lower total levels of serum cholesterol, low density lipoproteins (LDLs), very low density lipoproteins (VLDLs) and triglycerides. Deficiency of niacin can result in dermatitis, inflammation of the GI tract, the results of inadequate tryptophan. The RDI for niacin has been established to be 20 mg/day.

Pantothenic acid (Vitamin B₅) is essential in human nutrition for proper energy production, synthesis and breakdown of fatty acids, steroids, cholesterol, and amino acids, and functions as an antioxidant. The multiple functions of coenzyme A—important in oxidative phosphorylation—and acyl carrier protein, into which pantothenic acid is incorportated, are well recognized. The RDI for pantothenic acid has been established to be 10 mg/day.

Pyridoxine (Vitamin B₆) is important in the metabolism of proteins, fats and carbohydrates in the body. Vitamin B₆ supplementation has been found to lower systolic and diastolic pressure in hypertensive patients, protects vascular endothelial cells against platelet-induced damage and protects against atherosclerosis. Pyridoxine is known to be essential for the formation of hemoglobin and is important for utilization of stored glucose. The RDI for pyridoxine has been established to be 2 mg/day.

Vitamin B₁₂, a cobalt-containing enzyme cofactor, is necessary for normal cell growth and development notably in the development of red blood cells and is protective against neurodegenerative disorders in the body, especially the elderly. Vegetarians are susceptible to Vitamin B₁₂ deficiency. Insufficient intake of Vitamin B₁₂ may contribute to anemia. Vitamin B₁₂ may reduce the risk of atherosclerosis. The RDI for Vitamin B₁₂ has been established to be 6 μg/day.

Folic Acid (a B vitamin, sometimes referred to as vitamin B₉) is essential for proper cell growth and development, and for preventing neural birth defects. Folic acid deficiency can lead to anemia and deficiency of white blood cells, which play an important function in fighting off infectious disease. Folic acid has been shown to have anticarcinogenic actions and has a role in preventing cardiovascular disease, especially in the elderly. Insufficient intake of folate may contribute to anemia. Low levels of folate is one determinant of elevated homocysteine, along with genetic abnormality (a SNP, single nucleotide mutation), an important risk factor for atherosclerosis. The RDI for folate has been established to be 400 μg/day.

Biotin (a B vitamin, sometimes referred to as Vitamin H) is an enzyme cofactor involved in the biosynthesis of fats and carbohydrates, and metabolism of amino acids, in part due to its function in fixation of CO₂. Biotin supplementation has been found to improve glucose tolerance and decrease insulin resistance. The RDI for biotin has been established to be 300 μg/day.

Botanicals:

Lycopene is a carotenoid with potent antioxidant activity that protects cells against oxygen radicals and light damage. Research has shown than Lycopene can be protective against prostatic cancer and coronary heart disease. To date, no RDI has been established for Lycopene.

Rosemary is an herb that contains a mixture of bioflavonoids and potent antioxidants, including carnosol and carnosic acid. There is no RDI established for rosemary bioflavonoids, and there is no mammalian biosynthesis of these antioxidants.

Minerals:

Calcium is necessary for maintaining bone health and cell regulation. Calcium supplementation has been associated with reducing blood pressure in hypertensive patients as well as lowering serum cholesterol levels in man. The RDI for calcium has been established to be 1000 mg/day.

Chromium is an essential trace element that aids in regulating blood glucose by working with insulin to transport glucose into cells. Chromium works with insulin to convert carbohydrates and fat into energy. The RDI for chromium has been established to be 120 μg/day.

Iodine is an essential trace element that is vital to the function of the thyroid gland. Iodine is the essential component of thyroid hormones, which are crucial for normal development and controlling rates of metabolism. The RDI for iodine has been established to be 150 μg/day.

Magnesium is an essential mineral necessary for ATP production, and calcium regulation. Magnesium supplementation may have antihypertensive, glucose regulatory and cardioprotective actions in the body. Magnesium is essential for healthy nerve and muscle function and bone formation, and influences neuromuscular coordination. Magnesium may assist in preventing coronary heart disease. The RDI for magnesium has been established to be 400 mg/day.

Manganese is an essential trace element found in several key enzymes that are essential for normal cellular metabolism, and helps maintain protection against oxidative damage, controlling levels of and damage from reactive oxygen species. Manganese is required for glucose utilization, synthesis of mucopolysaccharides of cartilage, and biosynthesis of steroids. The RDI for manganese has been established to be 2 mg/day.

Molybdenum is an essential trace element needed for neurological and ocular health, and for processing many chemicals in the body that could otherwise be harmful, known to function as an enzyme cofactor in xanthine oxidase, important in metabolism of purine bases. The RDI for molybdenum has been established to be 75 μg/day.

Phosphorous is an essential mineral that is a central component of DNA, cellular membranes and energy production and storage within the cell. Phosphorous, in tandem with calcium, is essential to building and hardening of bones and teeth. The RDI for phosphorus has been established to be 1000 mg/day.

Potassium is an essential mineral that maintains intracellular tonicity and normal blood pressure, and has a primary role in transmission of neural signals in the body. Studies have shown that supplemental potassium may protect against strokes, cardiovascular disease, and other degenerative diseases. The DRV for potassium has been established to be 3500 mg/day.

Selenium is an essential trace element that acts in concert with Vitamins C and E to protect against oxidative damage in cells, and in particular selenium maintains the health of hepatic tissue. Selenium promotes cellular nerve growth and development, and cardiac health. As an enzyme cofactor, selenium is essential for healthy functioning of the heart muscle. The RDI for selenium has been established to be 70 μg/day.

### Other Considerations

The carotene, retinoid or combinations thereof, component (hereinafter referred to as “carotene(s)/retinoid(s)”) may be obtained from various sources such as vegetable and herbal sources, such as corn and leafy vegetables, and fermentation product sources available from the biotech industry. The carotenes/retinoids may also be synthesized by methods known in the art. Examples of carotenes include, but are not limited to, alpha-, beta-, gamma-, delta-, epsilon- and psi-carotene, and isomers thereof. Examples or retinoids include, but are not limited to, Vitamin A and Vitamin A analogs (e.g., retinoic acid). The carotene/retinoid purity and concentration in the various commercial sources will vary. For example, some sources may provide about a 1% w/w or less of carotene/retinoid in oil, or as an oil suspension, or in a protected dry form, e.g., a cobeadlet.

The concentrations of the xanthophylls and carotenes/retinoids in the formulations will vary, but will be in amounts useful in dietary supplements. In general, the combined concentration of xanthophylls and carotenes/retinoids in the formulations will be in the range of about 0.1 to 10% w/w. Preferred carotenoid concentrations, which are generally dependent on the selection of particular carotenes/retinoids and xanthophylls and their relative ratios, will be about 0.5 to 7% w/w. The individual concentrations of the xanthophylls and the carotenes/retinoids will not necessarily be the same. Preferred formulations for a general population of non-smokers will range from a concentration ratio from about 1:10 to about 10:1 of xanthophylls:carotenes/retinoids and the most preferred formulations will have concentration ratios ranging from about 2:1 to about 1:2 of xanthophylls:carotenes/retinoids. Preferred formulations for a population of smokers may range from 0% β-carotene to the RDA of β-carotene.

The most preferred formulations of the present invention include those in examples 1-4.

As stated above, the formulations will also contain one or more additional antioxidants. The antioxidants can be hydrophobic or hydrophilic. The antioxidants serve to inhibit the oxidative, photochemical and/or thermal degradation of the carotenoid components. Since antioxidants are also thought to be useful in nutritional health, they may also provide some nutritional benefit to the host. In general, the antioxidants will be natural antioxidants or agents derived therefrom. Examples of natural antioxidants and related derivatives include, but are not limited to, vitamin E and related derivatives, such as tocotrienols, alpha-, beta-, gamma-, delta- and epsilon-tocopherol, and their derivatives, such as the corresponding acetates, succinates; Vitamin C and related derivatives, e.g., ascorbyl palmitate; and natural oils, such as oil of rosemary. Preferred formulations will contain one or more hydrophobic antioxidants. The amount of antioxidant(s) contained in the formulation will be an amount effective to inhibit or reduce the oxidative, photochemical and/or thermal degradation of the carotenoid components. Such an amount is referred to herein as “an effective amount of one or more antioxidants.” In general, such an amount will range from about 0.1 to 10 times the amount of the xanthophyll and carotene/retinoid components and any other chemically sensitive components present, e.g., bioflavonoids. Preferred formulations, which will generally comprise about 0.5-25% w/w of carotenoids alone, or including bioflavonoids, will contain about 2 to 10% w/w of antioxidant. The antioxidants may be combined with designated nutrients in isolated reservoirs of cobeadlets before incorporation into the dosage form. Cobeadlets such as those described in U.S. Pat. Nos. 6,582,721, and 6,716,447, and in U.S. Patent Application Nos. 2005/0106272, and 2005/0147698, all of which are incorporated herein by reference, would be useful in the formulations of the present invention.

The formulations will also comprise one or more solidifying, bulking and agglomerating agents (collectively referred to herein as “solidifying agent(s)”). The solidifying agent(s) are used both in tableting and in generating solid-like carriers such as beadlets, capable of transforming oils into stable agglomerates suitable for granulation, blending, and compression required for tableting. Examples of solidifying agents useful in the preparation of the formulations include, but are not limited to, sucrose, glucose, fructose, starches (e.g., corn starch), syrups (e.g., corn syrup), and ionic and nonionic polymers including, but not limited to, PEGs and other poly ether-like alkoxy cellulosics (HPMC), gellan, carrageenans, Eucheuma gelatenae, guar, hyaluronates, alginates, chondroitin sulfate, pectins, and proteins, (e.g., collagen or their hydrolyzed products (e.g., gelatins or polypeptides)). Other solidifying agents known to those skilled in the art of dietary supplement preparation may also be used in the preparation of the formulations of the present invention. The amount of solidifying agent(s) will vary, depending on the other components contained in the formulation, but will generally comprise the majority weight and volume of the dietary supplement.

Optionally, the formulations of the present invention may also contain one or more bioflavonoids and/or glycosylated bioflavonoids. Bioflavonoids, or “flavonoids,” are flavone- and isoflavone-like structures found primarily in fruits and vegetables. Bioflavonoids are commercially available or may be synthesized by methods known in the art. Examples of bioflavonoids include, but are not limited to, quercetin, acacetin, liquiritin, rutin, taxifolin, nobiletin, tangeretin, apigenin, chyrsin, myricetin, genistein, daidzein, luteolin, naringenin, and kaempferol, and their derivatives, such as the corresponding methoxy-substituted analogs. The bioflavonoids may be useful in nutritional health as modulators of the rates of in vivo enzyme-mediated reactions. The bioflavonoids may also provide antioxidant activity and may be included in the formulations for this purpose.

Other oils may be present in the formulations of the present invention. The formulations will typically comprise an amount of vegetable oils or oleoresins, since the separate carotene/retinoid and/or xanthophyll components to be added to the formulations are generally commercially available as a diluted vegetable oil or oil suspension, or as an oleoresin extract. Such an amount of oil/oleoresin typically ranges from about 1 to 100 times the xanthophyll or carotene content in the formulation. For example, a xanthophyll extract to be included in a dietary supplement may contain 20% w/w lutein, 2% w/w zeaxanthin and 78% vegetable oil/oleoresin. Other oils may also be included in the formulations.

The formulations of the present invention may also comprise additional excipients useful in preparing and finishing the dietary supplements. Such excipients may include timed-release polymer coating agents useful in prolonging dissolution of the formulation in the digestive tract. Examples of such polymers include, but are not limited to ionic and nonionic polymers, such as PEGs and other poly ether-like alkoxy cellulosics (HPMC), gellan, carrageenans, Eucheuma gelatenae, starch, hyaluronates, chondroitin sulfate, pectins, and proteins, e.g., collagen. Since the xanthophyll/carotenes are highly pigmented, coating technology may be applied to the dietary supplement in order to provide a dietary supplement of uniform color. Examples of color coating agents may include, but are not limited to, polymers, colorants, sealants and surface active agents including, not limited to, fatty acids and esters, di- and triglycerides, phospholipids including mono- and di-alkyl glyceryl phosphates, nonionic agents (sugars, polysaccharides, e.g., HPMC and polysorbate 80) and ionic agents.

The above-described ingredients contained in the formulations may, in some cases, form microspheres within the dietary supplement. The dietary supplements may be of various size and shape.

The dietary supplements may be manufactured using a number of techniques known in the art. The ingredients described herein are preferably present in the dietary supplements of the invention in an amount sufficient to provide the daily dosage (amount consumed per day) when the recommended number of dietary supplements is ingested per day. It is critical, however, that the dietary supplement as described herein contain the described amounts of at least Vitamin C, Vitamin E, lutein, zeaxanthin, copper and zinc. β-carotene may or may not be present in preferred dietary supplements of the invention.

In some dosage forms, such as softgels, the use of concentrated oil phases of nutrients is desirable. These may be combined into a composite flowable core and concurrently protected with the aid of common diluents and antioxidants.

The following Examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### EXAMPLE 1

| | Amount | |
|---|---|---|
| | (per tab, capsule, | |
| | softgel, etc.) | |
| Ingredient | @ QID | |
| Beta Carotene | 0 | mg |
| Lutein | 1.67 | mg |
| Zeaxanthin | 0.83 | mg |
| Vitamin C | 128 | mg |
| Vitamin E | 107.5 | IU |
| Copper | 0.9 | mg |
| Zinc | 21.15 | mg |
| Vitamin D | 100 | IU |
| Vitamin K | 6.25 | μg |
| Vitamin B1 (thiamin) | 0.375 | mg |
| Vitamin B2 (riboflavin) | 2.5 | mg |
| Vitamin B3 (niacin) | 2.5 | mg |
| Vitamin B6 | 0.5 | mg |
| Folate/Folic Acid | 100 | μg |
| Vitamin B-12 | 1.5 | μg |
| Biotin | 7.5 | μg |
| Pantothenic Acid | 2.5 | mg |
| Phosphorus | 35 | mg |
| Iodine | 37.5 | μg |
| Calcium | 84 | mg |
| Magnesium | 25 | mg |
| Selenium | 10 | μg |
| Manganese | 0.5 | mg |
| Chromium | 30 | μg |
| Molybdenum | 18.75 | μg |
| Potassium | 20 | mg |
| Lycopene | 0.075 | mg |
| DHA | <25 | mg |
| Rosemary | >1 | mg |

| | |
|---|---|
| Water | TBD |

### EXAMPLE 2

| | Amount | |
|---|---|---|
| | (per tab, capsule, | |
| | softgel, etc.) | |
| Ingredient | @ QID | |
| Beta Carotene | 0.75 | mg |
| Lutein | 1.67 | mg |
| Zeaxanthin | 0.83 | mg |
| Vitamin C | 128 | mg |
| Vitamin E | 107.5 | IU |
| Copper | 0.9 | mg |
| Zinc | 21.15 | mg |
| Vitamin D | 100 | IU |
| Vitamin K | 6.25 | μg |
| Vitamin B1 (thiamin) | 0.375 | mg |
| Vitamin B2 (riboflavin) | 2.5 | mg |
| Vitamin B3 (niacin) | 2.5 | mg |
| Vitamin B6 | 0.5 | mg |
| Folate/Folic Acid | 100 | μg |
| Vitamin B-12 | 1.5 | μg |
| Biotin | 7.5 | μg |
| Pantothenic Acid | 2.5 | mg |
| Phosphorus | 35 | mg |
| Iodine | 37.5 | μg |
| Calcium | 84 | mg |
| Magnesium | 25 | mg |
| Selenium | 10 | μg |
| Manganese | 0.5 | mg |
| Chromium | 30 | μg |
| Molybdenum | 18.75 | μg |
| Potassium | 20 | mg |
| Lycopene | 0.075 | mg |
| DHA | <25 | mg |
| Rosemary | >1 | mg |

| | |
|---|---|
| Water | TBD |

### EXAMPLE 3

| | Amount | |
|---|---|---|
| Ingredient | (per tab) @ QID | |
| Beta Carotene | 0.75 | mg |
| Lutein | 2 | mg |
| Zeaxanthin | 1 | mg |
| Vitamin C | 125 | mg |
| Vitamin E | 100 | IU |
| Copper | 0.4 | mg |
| Zinc | 20 | mg |
| Vitamin D | 100 | IU |
| Vitamin K | 6.25 | μg |
| Vitamin B1 (thiamin) | 0.375 | mg |
| Vitamin B2 (riboflavin) | 2.5 | mg |
| Vitamin B3 (niacin) | 5 | mg |
| Vitamin B6 | 0.5 | mg |
| Folate/Folic Acid | 100 | μg |
| Vitamin B-12 | 1.5 | μg |
| Biotin | 7.5 | μg |
| Pantothenic Acid | 2.5 | mg |
| Phosphorus | 27.25 | mg |
| Iodine | 37.5 | μg |
| Magnesium | 25 | mg |
| Selenium | 17.5 | μg |
| Calcium | 84 | mg |
| Manganese | 0.5 | mg |
| Chromium | 30 | μg |
| Molybdenum | 18.75 | μg |
| Potassium | 20 | mg |
| Lycopene | 0.075 | mg |
| DHA | <25 | mg |
| Rosemary | >1 | mg |

| | |
|---|---|
| Water | TBD |

The following example, Example 4, indicates that observations may recommend a combination of different isomers of zeaxanthin, not just all of one or the other. Alternatively in Example 3 the inference would be correctly drawn to interpret the “zeaxanthin” to represent a ratio of zeaxanthins, from 0 to infinite, that is all of one or the other. The same inference would be accurate for the Examples in the other sets.

### EXAMPLE 4

| | Amount | |
|---|---|---|
| Ingredient | (per tab) @ QID | |
| Beta Carotene | 0.75 | mg |
| Lutein | 2 | mg |
| [R,R]-Zeaxanthin | 0.5 | mg |
| [R,S]-Zeaxanthin | 0.5 | mg |
| Vitamin C | 125 | mg |
| Vitamin E | 100 | IU |
| Copper | 0.4 | mg |
| Zinc | 20 | mg |
| Vitamin D | 100 | IU |
| Vitamin K | 6.25 | μg |
| Vitamin B1 (thiamin) | 0.5 | mg |
| Vitamin B2 (riboflavin) | 2.5 | mg |
| Vitamin B3 (niacin) | 5 | mg |
| Vitamin B6 | 0.5 | mg |
| Folate/Folic Acid | 100 | μg |
| Vitamin B-12 | 1.5 | μg |
| Biotin | 7.5 | μg |
| Pantothenic Acid | 2.5 | mg |
| Phosphorus | 25 | mg |
| Iodine | 37.5 | μg |
| Magnesium | 25 | mg |
| Selenium | 10 | μg |
| Calcium | 84 | mg |
| Manganese | 0.5 | mg |
| Chromium | 30 | μg |
| Molybdenum | 18.75 | μg |
| Potassium | 20 | mg |
| Lycopene | 0.075 | mg |
| DHA | <25 | mg |
| Rosemary | >1 | mg |

| | |
|---|---|
| Water | TBD |

The following example, Example 5, provides higher concentrations of carotenoids for individuals with low serum or pigment levels, or who might be less responsive to supplementation.

### EXAMPLE 5

| | Amount | |
|---|---|---|
| Ingredient | (per tab) @ QID | |
| Beta Carotene | 0.75 | mg |
| Lutein | 3.32 | mg |
| Zeaxanthin | 1.67 | mg |
| Vitamin C | 125 | mg |
| Vitamin E | 100 | IU |
| Copper | 0.4 | mg |
| Zinc | 20 | mg |
| Vitamin D | 100 | IU |
| Vitamin K | 6.25 | μg |
| Vitamin B1 (thiamin) | 0.5 | mg |
| Vitamin B2 (riboflavin) | 2.5 | mg |
| Vitamin B3 (niacin) | 5 | mg |
| Vitamin B6 | 0.5 | mg |
| Folate/Folic Acid | 100 | μg |
| Vitamin B-12 | 1.5 | μg |
| Biotin | 7.5 | μg |
| Pantothenic Acid | 2.5 | mg |
| Phosphorus | 25 | mg |
| Iodine | 37.5 | μg |
| Magnesium | 25 | mg |
| Selenium | 10 | μg |
| Calcium | 84 | mg |
| Manganese | 0.5 | mg |
| Chromium | 30 | μg |
| Molybdenum | 18.75 | μg |
| Potassium | 20 | mg |
| Lycopene | 0.075 | mg |
| DHA | <25 | mg |
| Rosemary | >1 | mg |

| | |
|---|---|
| Water | TBD |

The compositions in Example 6 describe meaningful formulations for individuals interested in maintaining ocular health, but who have no difficulty of consuming large tablets (or other dosage forms). The composition and dosing regimen of Example 6 would be appropriate and sufficient for the daily supplement requirement of such patients.

### EXAMPLE 6

| | Amount | |
|---|---|---|
| Ingredient | (per tab) @ BID | |
| Beta Carotene | 1.5 | mg |
| Lutein | 2 | mg |
| Zeaxanthin | 1 | mg |
| Vitamin C | 200 | mg |
| Vitamin E | 75 | IU |
| Copper | 0.5 | mg |
| Zinc | 20 | mg |
| Vitamin D | 200 | IU |
| Vitamin K | 12.5 | μg |
| Vitamin B1 (thiamin) | 0.751 | mg |
| Vitamin B2 (riboflavin) | 5.0 | mg |
| Vitamin B3 (niacin) | 10 | mg |
| Vitamin B6 | 1 | mg |
| Folate/Folic Acid | 200 | μg |
| Vitamin B-12 | 3.0 | μg |
| Biotin | 15 | μg |
| Pantothenic Acid | 5 | mg |
| Phosphorus | 70 | mg |
| Iodine | 75 | μg |
| Calcium | 168 | mg |
| Magnesium | 50 | mg |
| Selenium | 35 | μg |
| Manganese | 1 | mg |
| Chromium | 60 | μg |
| Molybdenum | 37.5 | μg |
| Potassium | 40 | mg |
| Lycopene | 0.150 | mg |
| DHA | <50 | mg |
| Rosemary | >1 | mg |

| | |
|---|---|
| Water | TBD |

Like the composition in Example 6, the composition in Examples 7 describes a meaningful formulation for individuals interested in maintaining ocular health yet who have a need for higher levels of carotenoids because either their serum or pigment levels are low. The composition and dosing regimen of Example 7 would be appropriate and sufficient for the daily supplement requirement of such patients.

### EXAMPLE 7

| | Amount | |
|---|---|---|
| Ingredient | (per tab) @ BID | |
| Beta Carotene | 1.5 | mg |
| Lutein | 6.7 | mg |
| Zeaxanthin | 3.3 | mg |
| Vitamin C | 200 | mg |
| Vitamin E | 100 | IU |
| Copper | 0.5 | mg |
| Zinc | 20 | mg |
| Vitamin D | 200 | IU |
| Vitamin K | 12.5 | μg |
| Vitamin B1 (thiamin) | 0.751 | mg |
| Vitamin B2 (riboflavin) | 5.0 | mg |
| Vitamin B3 (niacin) | 10 | mg |
| Vitamin B6 | 1 | mg |
| Folate/Folic Acid | 200 | μg |
| Vitamin B-12 | 3.0 | μg |
| Biotin | 15 | μg |
| Pantothenic Acid | 5 | mg |
| Phosphorus | 70 | mg |
| Iodine | 75 | μg |
| Calcium | 168 | mg |
| Magnesium | 50 | mg |
| Selenium | 35 | μg |
| Manganese | 1 | mg |
| Chromium | 60 | μg |
| Molybdenum | 37.5 | μg |
| Potassium | 40 | mg |
| Lycopene | 0.150 | mg |
| DHA | <50 | mg |
| Rosemary | >1 | mg |

| | |
|---|---|
| Water | TBD |

The compositions in Example 8 describe meaningful formulations for individuals interested in maintaining ocular health, yet whose diet does not require the supplementation with a multivitamin. The composition and dosing regimen of Example 6 would be appropriate and sufficient for the daily supplement requirement of such patients.

### EXAMPLE 8

| | Amount | |
|---|---|---|
| Ingredient | (per tab) @ BID | |
| Beta Carotene | 1.5 | mg |
| Lutein | 3.3 | mg |
| Zeaxanthin | 1.7 | mg |
| Vitamin C | 200 | mg |
| Vitamin E | 75 | IU |
| Copper | 0.5 | mg |
| Zinc | 20 | mg |
| Vitamin B2 (riboflavin) | 5.0 | mg |
| Folate/Folic Acid | 100 | μg |
| Vitamin B-12 | 3.0 | μg |
| Selenium | 20 | μg |
| Manganese | 5.0 | mg |
| Lycopene | 0.075 | mg |
| DHA | 12.5 | mg |
| Rosemary | >1 | mg |

| | |
|---|---|
| Water | TBD |

The composition in Example 9 describes a meaningful formulation for individuals interested in maintaining ocular health, but have a greater need for the xanthophylls. This composition and dosing regimen would be appropriate and sufficient for the daily supplement requirement of such patients.

### EXAMPLE 9

| | Amount | |
|---|---|---|
| Ingredient | (per tab) @ BID | |
| Beta Carotene | 1.5 | mg |
| Lutein | <13.2 | mg |
| Zeaxanthin | <6.7 | mg |
| Vitamin C | <200 | mg |
| Vitamin E | <200 | IU |
| Copper | 0.5 | mg |
| Zinc | 20 | mg |
| Vitamin B2 (riboflavin) | 5.0 | mg |
| Folate/Folic Acid | 100 | μg |
| Vitamin B-12 | 3.0 | μg |
| Selenium | 20 | μg |
| Manganese | 5.0 | mg |
| Lycopene | 0.075 | mg |
| DHA | 12.5 | mg |
| Rosemary | >1 | mg |

| | |
|---|---|
| Water | TBD |

### EXAMPLE 10

An AREDS Softgel—Minimum levels assuming NHANES normal intake & softgels, i.e., a supplement to a normal diet.

| | Amount | |
|---|---|---|
| | (per tab, capsule, | |
| | softgel, etc.) | |
| Ingredient | @ BID | |
| Vitamin A (beta carotene) | 5 | mg |
| Vitamin C | 200 | mg |
| Vitamin E | 110 | IU |
| Copper | 0.5 | mg |
| Zinc | 34 | mg |
| Omega-3 Fatty Acids | 33 | mg |
| (EPA/DHA) | | |
| Lutein (optional) | 3.3 | mg |
| Zeaxanthin (optional) | 1.7 | mg |
| Rosemary (optional) | >1 | mg |

### EXAMPLE 11

| | Amount | |
|---|---|---|
| | (per tab, capsule, | |
| | softgel, etc.) | |
| Ingredient | @ QID | |
| Lutein | 1.67 | mg |
| Zeaxanthin | 0.83 | mg |
| Vitamin A (beta carotene) | 2.8 | mg |
| Vitamin C | 86 | mg |
| Vitamin E | 90 | IU |
| Copper | 0. | mg |
| Zinc | 21.15 | mg |
| Vitamin D | 50 | IU |
| Vitamin K | 6.25 | μg |
| Vitamin B1 (thiamin) | 0.25 | mg |
| Vitamin B2 (riboflavin) | 0.25 | mg |
| Vitamin B3 (niacin) | 2.5 | mg |
| Vitamin B6 | 0.25 | mg |
| Folate/Folic Acid | 30 | μg |
| Vitamin B-12 | 0.25 | μg |
| Biotin | 7.5 | μg |
| Pantothenic Acid | 2.5 | mg |
| Phosphorus | | mg |
| Iodine | 37.5 | μg |
| Calcium | | mg |
| Magnesium | 25 | mg |
| Selenium | 25 | μg |
| Manganese | 0.5 | mg |
| Chromium | 30 | μg |
| Molybdenum | 18 | μg |
| Potassium | | mg |
| Lycopene | 0.075 | mg |
| DHA/EPA | 25 | mg |
| Rosemary | >1 | mg |

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. More specifically, it will be apparent that certain agents which are both chemically and structurally related may be substituted for the agents described herein to achieve similar results. All such substitutions and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.

United States Patents and Published Applications
U.S. Pat. No. 3,998,753
U.S. Pat. No. 4,254,100
U.S. Pat. No. 4,657,928
U.S. Pat. No. 4,670,247
U.S. Pat. No. 6,582,721
U.S. Pat. No. 6,716,447
20030064133
20050106272
20050147698

Books
Berman, BIOCHEMISTRY OF THE EYE, (Plenum, 1991).
Chaney TEXTBOOK OF BIOCHEMISTRY WITH CLINICAL CORRELATIONS, John Wiley & Sons, pp. 970-1 (1986)

Other Publications
Beem J BIOL CHEM 249:7298 (1974)
Bernstein et al., Retinal Tubulin Binds Macular Carotenoids, INV OPHTHAL & VIS SCI 38(1):167-175 (1997).
Bernstein et al. Identification and Characterization of Pi Isoform of Glutathione S-Transferase (GSTP1) as a Zeaxanthin-binding Protein in the Macula of the Eye, J. BIOL. CHEM 279(47):49447-49454 (2004)
Bone and Landrum et al. Analysis of Macular Pigment by HPLC: Retinal Distribution and Age Study INV. OPHTH VIS SCI 29:843-849 (1988).
Bone and Landrum et al. Macular Pigment in Donor Eyes with and without AMD: a Case-control Study, INV. OPHTH VIS SCI 42:235-240 (2001).
Chandler et al., J. BIOL. CHEM 261:928-33 (1986)
Chatterjee ARCH, OPHTHALMOL 56:756-60 (1956)
Fischer J NUTRITION 113:462-9 (1983)
Hammond et al., Sex differences in macular pigment optical density: relation to plasma carotenoid concentrations and dietary patterns, VISION RESEARCH 36:2001-2012 (1996a).
Hammond et al., Cigarette smoking and retinal carotenoids: implications for age-related macular degeneration, VISION RESEARCH 36:3003-3009 (1996b).
Hammond et al., Dietary modification of human macular pigment density, INV OPHTHAL & VIS SCI 38(9):1795-1801 (1997).
Handelman et al., Biological control of primate macular pigment: biochemical and densitometric studies, INV OPHTHAL & VIS SCI 32(2):257-267 (1991).
Harris, NATURE 132:27-8 (1993)
Hooper, JAMA 244:1960-1 (1980)
Jacques et al., Antioxidant status in persons with and without senile cataract, ARCH. OPHTHALM. 106:337 (1988).
Karcioglu SURV OPHTHALMOL 27:114-22 (1982)
Krinsky et al., Biologic mechanism of the protective role of lutein and zeaxanthin in the eye, ANNUAL REV NUTR 23:171-201 (2003)
Leure-duPree, RETINA 2:294-302 (1982a)
Leure-duPree, INVEST OPHTHALMOL VIS SCI 23:425-34 (1982b)
Machlin et al., Free radical tissue damage: protective role of antioxidant nutrients, FASEB J 1:441-445 (1987).
Newsome, D. A., Oral zinc in macular degeneration, ARCH. OPHTHALMOL. 106:192-198 (1988).
Ohrloff GRAEFE'S ARCH CLIN EXP OPHTHALMOL 222:79-81 (1984)
Orten, HUMAN BIOCHEMISTRY 10^{th} Edition, CV Mosby Co., p. 756 (1982)
Pennington J AM DIETETIC ASSOC 86:876-91 (1986)
Purcell ARCH, OPHTHALMOL 51:1-6 (1968)
Ringvold ACTA, OPHTHALMOLOGICA 63:227-80 (1985)
Russell ANN INT MED 99:227-39 (1983)
Seddon et al., Dietary carotenoids, vitamins a, c and e, and advanced age-related macular degeneration, JAMA 272(8):1413-1420 (1994).
Seddon and Willett et al., Prospective study of dietary fat and the risk of age-related macular degeneration, AM J CLIN NUTR 73:209-218 (2001).
Snodderly, Evidence for protection against age-related macular degeneration by carotenoids and antioxidant vitamins AM J CLIN NUTR 62(suppl):1448-1461S (1995).
Spector et al., EXP. EYE RES. 33:673 (1981).
Swanson BIOCHEM BIPHY RES COMM 45:1488-96 (1971)
Taylor, NUTRITIONAL AND ENVIRONMENTAL INFLUENCES ON THE EYE, (CRC, 1999).
Van Campen J NUTRITION 97:104-8 (1970)
Varma OPHTHALMIC RES 9:421-31 (1977)
Wagner GERIATRICS 40:111-25 (1985)
Williams PEDIAT RES 1:823 (1977)

We claim:

1. A dietary supplement comprising, approximately 0.03 wt. % to 0.3 wt. % copper, approximately 0.2 wt. % to 4 wt. % zinc, and Vitamin C, Vitamin E, and active carotenoid in the form of Vitamin A, lutein and/or zeaxanthin, wherein the concentration of Vitamin C in the dietary supplement is approximately 10 wt. % to 30 wt. % by weight, the concentration of Vitamin E in the dietary supplement is approximately 0.5 wt. % to 25 wt. % Vitamin E, and approximately 0 wt. % to 30 wt. % by weight of active carotenoid in the form of Vitamin A, lutein and/or zeaxanthin, and further comprising at least two or more compounds selected from the group consisting of vitamin K, vitamin B1, vitamin B2, vitamin B3, vitamin B6, folate, vitamin B12, Biotin, Pantothenic Acid, Phosphorus, Iodine, Magnesium, Selenium, Manganese, Chromium, Molybdenum, Potassium, Lycopene, docosahexaenoic acid (DHA), and Rosemary.

2. The dietary supplement of claim 1, wherein the Vitamin A is in the form of β-carotene.

3. The dietary supplement of claim 1, comprising:

| | Amount | |
|---|---|---|
| | (per tab, capsule, | |
| | softgel, etc.) | |
| Ingredient | @ QID | |
| Lutein | 1.67 | mg |
| Zeaxanthin | 0.83 | mg |
| Vitamin C | 128 | mg |
| Vitamin E | 107.5 | IU |
| Copper | 0.9 | mg |
| Zinc | 21.15 | mg |
| Vitamin D | 100 | IU |
| Vitamin K | 6.25 | μg |
| Vitamin B1 (thiamin) | 0.375 | mg |
| Vitamin B2 (riboflavin) | 2.5 | mg |
| Vitamin B3 (niacin) | 2.5 | mg |
| Vitamin B6 | 0.5 | mg |
| Folate/Folic Acid | 100 | μg |
| Vitamin B-12 | 1.5 | μg |
| Biotin | 7.5 | μg |
| Pantothenic Acid | 2.5 | mg |
| Phosphorus | 35 | mg |
| Iodine | 37.5 | μg |
| Calcium | 84 | mg |
| Magnesium | 25 | mg |
| Selenium | 10 | μg |
| Manganese | 0.5 | mg |
| Chromium | 30 | μg |
| Molybdenum | 18.75 | μg |
| Potassium | 20 | mg |
| Lycopene | 0.075 | mg |
| DHA | <25 | mg |
| Rosemary | >1 | mg |

wherein four supplements are ingested per day to provide the daily dosage amount.

4. The dietary supplement of claim 1, comprising:

| | Amount | |
|---|---|---|
| | (per tab, capsule, | |
| | softgel, etc.) | |
| Ingredient | @ QID | |
| Beta Carotene | 0.75 | mg |
| Lutein | 1.67 | mg |
| Zeaxanthin | 0.83 | mg |
| Vitamin C | 128 | mg |
| Vitamin E | 107.5 | IU |
| Copper | 0.9 | mg |
| Zinc | 21.15 | mg |
| Vitamin D | 100 | IU |
| Vitamin K | 6.25 | μg |
| Vitamin B1 (thiamin) | 0.375 | mg |
| Vitamin B2 (riboflavin) | 2.5 | mg |
| Vitamin B3 (niacin) | 2.5 | mg |
| Vitamin B6 | 0.5 | mg |
| Folate/Folic Acid | 100 | μg |
| Vitamin B-12 | 1.5 | μg |
| Biotin | 7.5 | μg |
| Pantothenic Acid | 2.5 | mg |
| Phosphorus | 35 | mg |
| Iodine | 37.5 | μg |
| Calcium | 84 | mg |
| Magnesium | 25 | mg |
| Selenium | 10 | μg |
| Manganese | 0.5 | mg |
| Chromium | 30 | μg |
| Molybdenum | 18.75 | μg |
| Potassium | 20 | mg |
| Lycopene | 0.075 | mg |
| DHA | <25 | mg |
| Rosemary | >1 | mg |

wherein four supplements are ingested per day to provide the daily dosage amount.

5. The dietary supplement of claim 1, comprising:

| | Amount | |
|---|---|---|
| | (per tab, capsule, | |
| | softgel, etc.) | |
| Ingredient | @ QID | |
| Beta Carotene | 0.75 | mg |
| Lutein | 2 | mg |
| Zeaxanthin | 1 | mg |
| Vitamin C | 125 | mg |
| Vitamin E | 100 | IU |
| Copper | 0.4 | mg |
| Zinc | 20 | mg |
| Vitamin D | 100 | IU |
| Vitamin K | 6.25 | μg |
| Vitamin B1 (thiamin) | 0.375 | mg |
| Vitamin B2 (riboflavin) | 2.5 | mg |
| Vitamin B3 (niacin) | 5 | mg |
| Vitamin B6 | 0.5 | mg |
| Folate/Folic Acid | 100 | μg |
| Vitamin B-12 | 1.5 | μg |
| Biotin | 7.5 | μg |
| Pantothenic Acid | 2.5 | mg |
| Phosphorus | 27.25 | mg |
| Iodine | 37.5 | μg |
| Magnesium | 25 | mg |
| Selenium | 17.5 | μg |
| Calcium | 84 | mg |
| Manganese | 0.5 | mg |
| Chromium | 30 | μg |
| Molybdenum | 18.75 | μg |
| Potassium | 20 | mg |
| Lycopene | 0.075 | mg |
| DHA | <25 | mg |
| Rosemary | >1 | mg |

wherein four supplements are ingested per day to provide the daily dosage amount.

6. The dietary supplement of claim 1, comprising:

| | Amount | |
|---|---|---|
| | (per tab, capsule, | |
| | softgel, etc.) | |
| Ingredient | @ QID | |
| Beta Carotene | 0.75 | mg |
| Lutein | 2 | mg |
| [R,R]-Zeaxanthin | 0.5 | mg |
| [R,S]-Zeaxanthin | 0.5 | mg |
| Vitamin C | 125 | mg |
| Vitamin E | 100 | IU |
| Copper | 0.4 | mg |
| Zinc | 20 | mg |
| Vitamin D | 100 | IU |
| Vitamin K | 6.25 | μg |
| Vitamin B1 (thiamin) | 0.5 | mg |
| Vitamin B2 (riboflavin) | 2.5 | mg |
| Vitamin B3 (niacin) | 5 | mg |
| Vitamin B6 | 0.5 | mg |
| Folate/Folic Acid | 100 | μg |
| Vitamin B-12 | 1.5 | μg |
| Biotin | 7.5 | μg |
| Pantothenic Acid | 2.5 | mg |
| Phosphorus | 25 | mg |
| Iodine | 37.5 | μg |
| Calcium | 84 | mg |
| Magnesium | 25 | mg |
| Selenium | 10 | μg |
| Manganese | 0.5 | mg |
| Chromium | 30 | μg |
| Molybdenum | 18.75 | μg |
| Potassium | 20 | mg |
| Lycopene | 0.075 | mg |
| DHA | <25 | mg |
| Rosemary | >1 | mg |

wherein four supplements are ingested per day to provide the daily dosage amount.

7. The dietary supplement of claim 1, comprising:

| | Amount | |
|---|---|---|
| | (per tab, capsule, | |
| | softgel, etc.) | |
| Ingredient | @ QID | |
| Beta Carotene | 0.75 | mg |
| Lutein | 3.32 | mg |
| Zeaxanthin | 1.67 | mg |
| Vitamin C | 125 | mg |
| Vitamin E | 100 | IU |
| Copper | 0.4 | mg |
| Zinc | 20 | mg |
| Vitamin D | 100 | IU |
| Vitamin K | 6.25 | μg |
| Vitamin B1 (thiamin) | 0.5 | mg |
| Vitamin B2 (riboflavin) | 2.5 | mg |
| Vitamin B3 (niacin) | 5 | mg |
| Vitamin B6 | 0.5 | mg |
| Folate/Folic Acid | 100 | μg |
| Vitamin B-12 | 1.5 | μg |
| Biotin | 7.5 | μg |
| Pantothenic Acid | 2.5 | mg |
| Phosphorus | 25 | mg |
| Iodine | 37.5 | μg |
| Magnesium | 25 | mg |
| Selenium | 10 | μg |
| Calcium | 84 | mg |
| Manganese | 0.5 | mg |
| Chromium | 30 | μg |
| Molybdenum | 18.75 | μg |
| Potassium | 20 | mg |
| Lycopene | 0.075 | mg |
| DHA | <25 | mg |
| Rosemary | >1 | mg |

wherein four supplements are ingested per day to provide the daily dosage amount.

8. The dietary supplement of claim 1, comprising:

| | Amount | |
|---|---|---|
| | (per tab, capsule, | |
| | softgel, etc.) | |
| Ingredient | @ BID | |
| Beta Carotene | 1.5 | mg |
| Lutein | 2 | mg |
| Zeaxanthin | 1 | mg |
| Vitamin C | 200 | mg |
| Vitamin E | 75 | IU |
| Copper | 0.5 | mg |
| Zinc | 20 | mg |
| Vitamin D | 200 | IU |
| Vitamin K | 12.5 | μg |
| Vitamin B1 (thiamin) | 0.751 | mg |
| Vitamin B2 (riboflavin) | 5.0 | mg |
| Vitamin B3 (niacin) | 10 | mg |
| Vitamin B6 | 1 | mg |
| Folate/Folic Acid | 200 | μg |
| Vitamin B-12 | 3.0 | μg |
| Biotin | 15 | μg |
| Pantothenic Acid | 5 | mg |
| Phosphorus | 70 | mg |
| Iodine | 75 | μg |
| Calcium | 168 | mg |
| Magnesium | 50 | mg |
| Selenium | 35 | μg |
| Manganese | 1 | mg |
| Chromium | 60 | μg |
| Molybdenum | 37.5 | μg |
| Potassium | 40 | mg |
| Lycopene | 0.150 | mg |
| DHA | <50 | mg |
| Rosemary | >1 | mg |

wherein two supplements are ingested per day to provide the daily dosage amount.

9. The dietary supplement of claim 1, comprising:

| | Amount | |
|---|---|---|
| | (per tab, capsule, | |
| | softgel, etc.) | |
| Ingredient | @ BID | |
| Beta Carotene | 1.5 | mg |
| Lutein | 6.7 | mg |
| Zeaxanthin | 3.3 | mg |
| Vitamin C | 200 | mg |
| Vitamin E | 100 | IU |
| Copper | 0.5 | mg |
| Zinc | 20 | mg |
| Vitamin D | 200 | IU |
| Vitamin K | 12.5 | μg |
| Vitamin B1 (thiamin) | 0.751 | mg |
| Vitamin B2 (riboflavin) | 5.0 | mg |
| Vitamin B3 (niacin) | 10 | mg |
| Vitamin B6 | 1 | mg |
| Folate/Folic Acid | 200 | μg |
| Vitamin B-12 | 3.0 | μg |
| Biotin | 15 | μg |
| Pantothenic Acid | 5 | mg |
| Phosphorus | 70 | mg |
| Iodine | 75 | μg |
| Calcium | 168 | mg |
| Magnesium | 50 | mg |
| Selenium | 35 | μg |
| Manganese | 1 | mg |
| Chromium | 60 | μg |
| Molybdenum | 37.5 | μg |
| Potassium | 40 | mg |
| Lycopene | 0.150 | mg |
| DHA | <50 | mg |
| Rosemary | >1 | mg |

wherein two supplements are ingested per day to provide the daily dosage amount.

10. The dietary supplement of claim 1, comprising:

| | Amount | |
|---|---|---|
| | (per tab, capsule, | |
| | softgel, etc.) | |
| Ingredient | @ BID | |
| Beta Carotene | 1.5 | mg |
| Lutein | 3.3 | mg |
| Zeaxanthin | 1.7 | mg |
| Vitamin C | 200 | mg |
| Vitamin E | 75 | IU |
| Copper | 0.5 | mg |
| Zinc | 20 | mg |
| Vitamin B2 (riboflavin) | 5.0 | mg |
| Folate/Folic Acid | 100 | μg |
| Vitamin B-12 | 3.0 | μg |
| Selenium | 20 | μg |
| Manganese | 5.0 | mg |
| Lycopene | 0.075 | mg |
| DHA | 12.5 | mg |
| Rosemary | >1 | mg |

wherein two supplements are ingested per day to provide the daily dosage amount.

11. The dietary supplement of claim 1, comprising:

| | Amount | |
|---|---|---|
| | (per tab, capsule, | |
| | softgel, etc.) | |
| Ingredient | @ BID | |
| Beta Carotene | 1.5 | mg |
| Lutein | <13.2 | mg |
| Zeaxanthin | <6.7 | mg |
| Vitamin C | <200 | mg |
| Vitamin E | <200 | IU |
| Copper | 0.5 | mg |
| Zinc | 20 | mg |
| Vitamin B2 (riboflavin) | 5.0 | mg |
| Folate/Folic Acid | 100 | μg |
| Vitamin B-12 | 3.0 | μg |
| Selenium | 20 | μg |
| Manganese | 5.0 | mg |
| Lycopene | 0.075 | mg |
| DHA | 12.5 | mg |
| Rosemary | >1 | mg |

wherein two supplements are ingested per day to provide the daily dosage amount.

12. The dietary supplement of claim 1, comprising

| | Amount | |
|---|---|---|
| | (per tab, capsule, | |
| | softgel, etc.) | |
| Ingredient | @ BID | |
| Vitamin A (beta carotene) | 5 | mg |
| Vitamin C | 200 | mg |
| Vitamin E | 110 | IU |
| Copper | 0.5 | mg |
| Zinc | 34 | mg |
| Omega-3 Fatty Acids | 33 | mg |
| (EPA/DHA) | | |
| Lutein (optional) | 3.3 | mg |
| Zeaxanthin (optional) | 1.7 | mg |
| Rosemary (optional) | >1 | mg |

wherein the supplement is a softgel and wherein two supplements are ingested per day to provide the daily dosage amount.

13. The dietary supplement of claim 1, comprising

| | Amount | |
|---|---|---|
| | (per tab, capsule, | |
| | softgel, etc.) | |
| Ingredient | @ QID | |
| Lutein | 1.67 | mg |
| Zeaxanthin | 0.83 | mg |
| Vitamin A (beta carotene) | 2.8 | mg |
| Vitamin C | 86 | mg |
| Vitamin E | 90 | IU |
| Copper | 0. | mg |
| Zinc | 21.15 | mg |
| Vitamin D | 50 | IU |
| Vitamin K | 6.25 | μg |
| Vitamin B1 (thiamin) | 0.25 | mg |
| Vitamin B2 (riboflavin) | 0..25 | mg |
| Vitamin B3 (niacin) | 2.5 | mg |
| Vitamin B6 | 0.25 | mg |
| Folate/Folic Acid | 30 | μg |
| Vitamin B-12 | 0.25 | μg |
| Biotin | 7.5 | μg |
| Pantothenic Acid | 2.5 | mg |
| Phosphorus | | mg |
| Iodine | 37.5 | μg |
| Calcium | | mg |
| Magnesium | 25 | mg |
| Selenium | 25 | μg |
| Manganese | 0.5 | mg |
| Chromium | 30 | μg |
| Molybdenum | 18 | μg |
| Potassium | | mg |
| Lycopene | 0.075 | mg |
| DHA/EPA | 25 | mg |
| Rosemary | >1 | mg |

wherein the supplement is a softgel and wherein four supplements are ingested per day to provide the daily dosage amount.
